(19) United States
(12) Patent Application Publication
(10) Pub. No.: US 2025/0032690 A1
(43) Pub. Date: Jan. 30, 2025

(54) METHOD AND SYSTEM FOR PROVIDING ON-LINE QUALITY INJECTIBLE PERITONEAL DIALYSATE AT HIGH FLOW RATES

(71) Applicant: Byonyks Medical Devices, Inc., Itasca, IL (US)

(72) Inventor: Rodney S. Kenley, Itasca, IL (US)

(21) Appl. No.: 18/716,397

(22) PCT Filed: Dec. 22, 2022
(86) PCT No.: PCT/US2022/082269

§ 371 Date: Jun. 4, 2024

Related U.S. Application Data
(60) Provisional application No. 63/293,257, filed on Dec. 23, 2021.

(51) Int. Cl. A61M 1/28

(52) U.S. Cl. CPC A61M 1/287; A61M 2205/50

(57) ABSTRACT

A system includes an apparatus configured to mix water, an acid/electrolyte concentrate, an osmotic concentrate, and a neutralizing buffer to produce a supply dialysate, a supply chamber configured to store the supply dialysate, a first filter that is configured to receive the supply dialysate from the supply chamber and to remove contaminants from the supply dialysate, a second filter, and one or more valves that: in a first configuration allow the second filter to receive a spent dialysate from the patient drain line to capture biological components from the spent dialysate such that the spent dialysate is dispensed via the system drain line, and in a second configuration allow the second filter to receive the supply dialysate to remove further contaminants from the supply dialysate such that the supply dialysate and the biological components are dispensed via the patient fill line.

### CROSS-REFERENCE TO RELATED APPLICATIONS

This application claims priority to U.S. Provisional Patent Application No. 63/293,257, filed on Dec. 23, 2021, the entire contents of which are incorporated by reference herein.

### BACKGROUND

There are two principal dialysis methods used to support patients requiring renal replacement therapy: hemodialysis and peritoneal dialysis. Peritoneal dialysis utilizes the patient's own peritoneum as a semipermeable membrane. The peritoneum is the membranous lining of the body cavity surrounding all of the organs between the diaphragm and the pelvis that, due to the large number of blood vessels and capillaries imbedded therein, is capable of acting as a natural semipermeable membrane.

In peritoneal dialysis, a sterile dialysate is infused into the peritoneal cavity by way of an indwelling catheter. This can be accomplished manually by gravity or with the use of a machine known as a cycler. The dialysate is allowed to dwell in the peritoneal cavity for a sufficient length of time (e.g. 4 hours) to yield a net removal of toxins and water after which the dialysate is drained and replaced with fresh dialysate.

Whereas in hemodialysis, fluid removal is accomplished by generating a transmembrane pressure with the aid of the hemodialysis machine, this is not possible with peritoneal dialysis, necessitating a different strategy for eliminating the excess fluid from patients. In peritoneal dialysis, instead of creating a hydrostatic pressure gradient between blood and dialysate, an osmotic pressure gradient is generated by including an osmotic agent in the peritoneal dialysate. The osmotic agent used in the vast majority of peritoneal dialysate is glucose. Although, as discussed below, glucose presents significant problems in this application, over 40 years of research has produced no suitable practical alternative for use in daily exchanges.

The primary difficulty with including glucose in peritoneal dialysate is encountered during terminal sterilization (e.g. by autoclaving). Whereas any solution instilled into a sensitive body cavity like the peritoneum would ideally be at physiological pH (e.g. 7.2-7.4), it is not possible to terminally sterilize a glucose-containing solution at physiological pH. Peritoneal dialysate (as well as other pharmaceutical solutions containing glucose, e.g., Dextrose 5 water or Total Parenteral Nutrition) is universally terminally heat sterilized (i.e. autoclaved), typically at 120° C. for approximately 2 hours to insure complete inactivation of bacteria and their spores and also under pressure in order to prevent boiling. Under autoclaving conditions, were the pH of the solution maintained in the physiological range, the glucose in the solution would discolor (caramelize) and also convert into multiple reaction products known collectively as glucose degradation products (GDPs).

Several of these GDPs, such as 3-deoxyglucosone (3-DG), 3,4-dideoxyglucosone-3-ene (3,4-DGE), glyoxal, methylglyoxal, 5-hydroxymethylfurfural (5-HMF), 2-furaldehyde (2-FA), formaldehyde and acetaldehyde, have been identified in peritoneal dialysis fluids (PDFs). GDPs are highly reactive precursors of Advanced Glycation End products (AGEs) in proteins. AGEs result from a chemical reaction when reduced carbohydrates (such as glucose) react with amino acids or nucleotides. In 1912, Louis Maillard was the first to describe this non-enzymatic reaction known as the “Maillard reaction.” Among the GDPs cited above, 5-HMF and 2-FA are considered as important indicators of degradation and they may appear in the glucose degradation process under sterilizing conditions.

Although precise thresholds of toxicity are not yet known for GDPs and AGEs administered to chronically-ill patients, it has been shown that high levels of GDPs and AGEs have an impact on cell homeostasis, are involved in oxidative stress, are associated with cellular inhibition, induce apoptosis in human leukocytes and renal epithelial cells, cause degradation of mesothelial cells and peritoneal membrane characteristics, have an impact on the cardiovascular system, are associated with an increase in cardiovascular morbidity and a decline in renal function or cause kidney damage. Other studies have shown that the accumulation of AGEs in patients suffering from diabetes mellitus can lead to microvascular complications such as diabetic retinopathy or diabetic vascular complications. Very high levels of 5-HMF may lead to acute toxicity.

Consequently, it is necessary for manufacturers of PDFs to sterilize these solutions at sub-physiologic pH to minimize caramelization and formation of GDPs. However, it has also been shown that PDFs produced and instilled at lower pH levels result in a transient diminution of the meager host defenses resident in the peritoneal cavity which contributes to lowering a patient's ability to respond to a bacterial invasion which, in turn, leads to a higher incidence of peritonitis; one of the leading complications of peritoneal dialysis. Also, PDFs at low pH cause pain on infusion in many patients and contribute to patients electing to perform hemodialysis instead or to drop out of peritoneal dialysis after some tenure on the modality; all of which results in incremental costs to the clinical providers and suboptimal clinical outcomes for patients.

Therefore, the manufacturers of PDFs have had to strike a compromise between minimizing the formation of GDPs during sterilization and the complications caused by low pH. This compromise has resulted in the majority of PDFs being produced at a pH of 5.2-5.5 even though published investigations have determined that GDP production is only minimized once the pH is lowered to a range of 2.0-2.6 during sterilization (although solutions sterilized at 3.0 were only marginally higher in GDP concentration).

Within the past few years, several low-GDP PDFs, featuring a double-chamber bag system that allows for sterilization and storage of the glucose at a low pH, have become commercially available. The beneficial effects on cell viability and peritoneal host defenses of a neutral pH combined with a low concentration of GDPs are abundantly documented in the literature. However, PDFs manufactured in this way result in a product that requires incremental manipulation by the patient during each exchange, additional training of the patients, and increased cost to manufacture the product, all of which has led to poor adoption of these products by the clinical providers of dialysis therapy.

Another major disadvantage of peritoneal dialysis and a major reason why patients fail to be maintained on this therapy is inadequacy. Inadequacy can be manifested as either or both a failure to remove adequate amounts of uremic toxins or adequate amounts of water (ultrafiltration) from the patient.

There are two primary forms of peritoneal dialysis (PD): Continuous Ambulatory Peritoneal Dialysis (CAPD) and continuous Cycling Peritoneal Dialysis (CCPD; A.K.A. APD for Automated Peritoneal Dialysis).

With CAPD, patients infuse dialysate from a terminally sterilized plastic bag, through a transfer set and an indwelling catheter into their peritoneal cavity. The volume infused is adjusted to the size of the patient with 2 liters being most typical. However, the larger the patient, the more toxins and water must be removed so the infusion volume must be increased, sometimes up to 3 liters.

Once the solution is infused, it is allowed to dwell in the peritoneal cavity while the toxins and water migrate out of the blood. Atypical dwell time is 4 hours. Toxin and water removal is fastest at the beginning of a dwell when the concentration gradient between blood and dialysate is greatest. The rate of removal approaches zero as the blood and dialysate reach equilibrium. At this point, a significant amount of glucose has been absorbed into the bloodstream.

After the dwell period, the patient drains the contents of their peritoneum back into the empty bag that was used for infusion, disconnects and discards it, attaches a new bag and initiates another fill/dwell/drain cycle. CAPD patients do this “exchange” either 4 or 5 times per day; one in the morning, one midday, one at dinner time, and one just before going to sleep. This last nighttime exchange will be indwelling for around 8 hours during which time much of the water that has been attracted into the peritoneum will be reabsorbed along with the glucose used to attract it unless a much higher concentration of glucose is employed, e.g., 4.25% vs 1.5% for daytime exchanges. This drastically increases the amount of glucose absorbed into the patient which translates into another major disadvantage of PD. About half of all dialysis patients contracted end-stage renal failure secondary to diabetes and loading up diabetics with sugar from the dialysate is problematic.

Just as problematic for any PD patient is the fact that they are absorbing a significant number of calories via the sugar uptake from the dialysate which reduces their appetite for protein and they can easily become malnourished, which is one of the reasons patients die or drop out of this therapy.

Exacerbating this protein intake insufficiency is the fact that amino acids and proteins also migrate out of the blood and into the dialysate and are eliminated from the body with each drain.

Joining the desirable amino acids and proteins that are lost in each drain are whatever components of host defense/immune response (e.g., macrophages, chemotaxis components) that might have migrated into the peritoneum to fight the ingress of any peritonitis-causing bacteria. This of course leads to increased cases of peritonitis.

Finally, CAPD dropout has two other causes inherent in the procedure. One is “burn-out” caused by the tedium of performing 4-5 exchanges every day for the rest of their life. The other is the relatively enormous volume of supplies that must be received and stored in the patients' homes every month.

With CCPD, the daytime exchanges are simply flipped into the 8 hours of sleep with the inflow and outflow of dialysate controlled by an instrument (cycler). This is designed to alleviate the patient from the drudgery of performing these exchanges during their waking hours. Typically, the cycler has left them with a final fill volume just before they wake up and this is left in the peritoneum until around lunch time when the patient performs a drain. The patient then has the option to remain “dry” until that night which has the benefits of letting their peritoneal membrane recover from the constant onslaught of low pH/high osmolarity solution, or, if they require more toxin and water removal, they can instill another fill volume for the rest of the day.

CCPD solves some of the disadvantages of CAPD but many remain. These disadvantages, which typically lead to drop-out (to hemodialysis) or death, can be summarized as follows:

Inadequacy: This is derived directly from the limit on how much dialysate is put through the patient in 24 hours, i.e. PD is limited by a low dialysate flow rate (abbreviated Q_{d}). Attempting to increase the Q_{d} with current techniques involves increasing the number of exchanges and/or the volume of dialysate. This would, in turn, increase burn-out, the storage space required, and increase the cost; all of which are mostly intolerable to both patients and providers.

Glucose uptake: This is highly undesirable for diabetics and leads to protein malnutrition. This results from: a) dwell times that allow the dialysate and blood to come to equilibrium (another ramification of low Q_{d}) and b) the use of 4.25% glucose during the long dwells.

Ultrafiltration failure and peritonitis: The low pH, high osmolarity, and presence of GDPs and AGEs contribute to scarring of the peritoneal membrane which reduces its permeability to water transmission, inactivates components of host defense, and increases inflammation.

Amino acid, protein, and white cell losses with each drain: This results from the spent dialysate being sent directly to drain. This contributes to protein malnutrition and a higher incidence of peritonitis.

Burn-out: This results from inescapable and burdensome daily CAPD exchange routine or cycler set-up/tear-down.

Burdensome volume of supplies in the home: This is mainly the result of performing PD with terminally sterilized bags of solution, their associated tubing sets and connectors, and the ancillary supplies required to perform aseptic connections/disconnections.

Accordingly, there exists a need to overcome the aforementioned disadvantages of peritoneal dialysis.

### SUMMARY

A first example includes a system for peritoneal dialysis, the system comprising: a mixing apparatus configured to mix water, an acid/electrolyte concentrate, an osmotic concentrate, and a neutralizing buffer to produce a supply dialysate; a supply chamber configured to store the supply dialysate; a first filter comprising a first port and a second port, wherein the first filter is configured to receive the supply dialysate from the supply chamber at the first port and to remove contaminants from the supply dialysate; a second filter comprising a third port and a fourth port; a patient drain line connected to the third port; a patient fill line connected to the third port; a system drain line connected to the fourth port; and one or more valves that: in a first configuration allow the second filter to receive a spent dialysate from the patient drain line at the third port to capture biological components from the spent dialysate such that the spent dialysate is dispensed via the system drain line, and in a second configuration allow the second filter to receive the supply dialysate from the second port at the fourth port to remove further contaminants from the supply dialysate such that the supply dialysate and the biological components are dispensed via the patient fill line.

A second example includes a method of operating the system of the first example, the method comprising: moving the spent dialysate into the third port and then through the second filter to capture the biological components from the spent dialysate; mixing, via the mixing apparatus, the water, the acid/electrolyte concentrate, the osmotic concentrate, and the neutralizing buffer to produce the supply dialysate; moving the supply dialysate into the first port to remove the contaminants from the supply dialysate; moving the supply dialysate from the second port into the fourth port and then through the second filter to remove the further contaminants from the supply dialysate; and dispensing the supply dialysate and the biological components from the third port via the patient fill line.

A third example includes a computer readable medium storing instructions that, when executed by the system of the first example, cause the system to perform the method of the second example.

A fourth example includes a system for peritoneal dialysis, the system comprising: a mixing apparatus configured to mix water, an acid/electrolyte concentrate, a osmotic concentrate, and a neutralizing buffer to produce a supply dialysate; a supply chamber configured to store the supply dialysate; a drain line; a filter; and one or more valves that in a first configuration allow the filter to receive a spent dialysate from the drain line at a first port of the filter to capture biological components within the spent dialysate and in a second configuration allow the filter to receive the supply dialysate from the supply chamber at a second port of the filter to remove contaminants from the supply dialysate.

A fifth example includes a method for conducting peritoneal dialysis, the method comprising: providing the system of the first example or the fourth example; mixing, via the mixing apparatus, the water, the acid/electrolyte concentrate, the osmotic concentrate, and the neutralizing buffer to produce the supply dialysate; moving the supply dialysate into the first port and through the first filter to remove the contaminants from the supply dialysate; moving the supply dialysate from the second port into the fourth port and then through the second filter to remove the further contaminants from the supply dialysate; moving the purified dialysate to a peritoneal cavity of the patient for peritoneal dialysis for a predetermined time while generating spent dialysate; moving the spent dialysate into the third port and then through the second filter to capture the biological components from the spent dialysate and generating depleted spent dialysate and draining the depleted spent dialysate; and repeating steps (ii) to (vi) for dispensing the supply dialysate for further peritoneal dialysis while dislodging the captured biological components from the second filter for introduction back to the patient.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a flow chart of a method, according to an example.

FIG. 2 is a block diagram of a computing device, according to an example.

FIG. 3 is a schematic diagram of a system in a configuration for capturing biological components from a spent dialysate, according to an example.

FIG. 4 is a schematic diagram of a system in a configuration for removing contaminants from a supply dialysate such that the supply dialysate and the captured biological components are dispensed via a patient fill line, according to an example.

FIG. 5 is a block diagram of a method, according to an example.

FIG. 6 is a block diagram of a method, according to an example.

### DETAILED DESCRIPTION

This disclosure includes examples that can help alleviate some disadvantages of previous systems and methods used for peritoneal dialysis. Conventional methods for peritoneal dialysis can leave the patient with depleted levels of proteins and host defenses (e.g., white blood cells, macrophages, and/or chemotaxis components) in their blood because some proteins and host defenses, along with metabolic waste products such as uremic toxins, can end up in the spent dialysate that that is drained from the patient. The loss of the host defenses can contribute to peritonitis and the loss of the proteins can contribute to malnutrition. As such, the patient would benefit by retaining the proteins and host defenses that are typically lost during peritoneal dialysis.

This disclosure provides examples of a peritoneal dialysis solution preparation and treatment system. The components included in the system and their function are delineated below roughly in the order they are first contacted by fluid flowing through the system as follows:

A three-way valve V1 at the inlet to the mixing apparatus allows water from a separate upstream water purification system to enter the mixing apparatus. This valve V1 is alternately used to allow fluid to be recirculated within all of the fluid paths of the device through the disinfection bypass fluid path between valves V1 and V16.

A first conductivity cell C1 downstream of the inlet is used to assure that the water being drawn into the mixing apparatus is at or below the safe upper limit of conductivity as set in a software lookup table in the system's memory.

A pressure sensor P1 downstream of the conductivity cell C1 is installed to assure that the water pressure presented to the inlet side of water pump PU1 is sufficiently high to prevent deaeration and cavitation.

A granular activated carbon cartridge (GAC) downstream of the pressure sensor P1 is used to remove any chlorine or chloramine that may have broken through the chlorine/chloramine removal apparatus of the separate water purification system supplying water to this device during a treatment. This component may or may not be necessary depending on the method of chlorine/chloramine removal implemented in the upstream water purification system.

An in-line fluid heater with two associated temperature sensors is placed downstream of the GAC. One sensor upstream (T1) and one sensor downstream (T2) of the heater, in combination with the on-board circuitry and software, are used to control the temperature of the water passed through the heater.

A water metering pump PU1 downstream of the heater precisely controls the flow of water to the downstream components of the system.

A proportioning pump PU2 downstream of the water metering pump PU1 precisely meters the amount of the acid/electrolyte concentrate according to the dialysate prescription residing in software.

A proportioning pump PU3 downstream of the pump PU2 precisely meters the amount of the neutralizing buffer according to the dialysate prescription residing in software.

A proportioning pump PU4 downstream of the pump PU3 precisely meters the amount of osmotic concentrate according to the dialysate prescription residing in software.

A flexible supply chamber is surrounded by a rigid enclosure that can be vented to atmosphere through valve V2. When vented through open valve V2, the supply chamber can be expanded to its maximum volume as limited by the physical dimensions of the rigid enclosure under the positive pressure created by pumps PU1, PU2, PU3, and PU4 as they deliver the constituents of the supply dialysate into the supply chamber so long as downstream valves V3 and V4 are oriented to prevent any outflow from this chamber. This supply chamber can also be used to deliver its contents downstream by closing valve V2 and opening valve V15 which allows positive air pressure generated by an air compressor (AC) and stored in the rigid positive pressure reservoir (PPR) to be delivered into the rigid enclosure surrounding the supply chamber. The contents of the supply chamber will then be delivered downstream so long as downstream valves under the control of the system software are correctly oriented to allow the outflow of the contents.

Redundant conductivity cells C2 and C3 downstream of the supply chamber where one is the primary sensor and the other located on the safety system side of the software to verify the primary are included. Their purpose is to insure that the dialysate is constituted in a safe range around the expected conductivity as located in a software lookup table for the dialysate prescription that has been set by the patient's physician and also loaded into the software.

Redundant temperature sensors T3 and T4 where, like the conductivity sensors, one is the primary and the other is the safety, are included. Their purpose is to assure that the dialysate being delivered to the patient is in a physiologically safe range.

Three-way valve V3 allows fluid to be directed either toward valve V5 (PF2) and V6 (drain) or into the first pyrogen filter PF1. PF1 and/or PF2 can take the form of a Medica DiaPure® filter and/or a Nephros® DSU-D filter.

A first pyrogen filter PF1 through which supply dialysate must pass before reaching a patient's peritoneal cavity is designed to remove any pyrogens and microorganisms from the supply dialysate. Air is removed from the hollow fibers of PF1 by opening valve V7 (to atmosphere) with valve V4 closed (to drain and PF2) while water is being perfused through its fiber bundle. This pushes the air past V7 and through a 0.2 micron sterilizing filter out to atmosphere. Air in the space between the fiber bundle and the rigid case encapsulating the fiber bundle is removed after the fiber bundle is primed by closing V7 and opening valve V4 which allows fluid to flow through the fibers, fill the case, and flow through the fluid path connecting the outflow port of PF1 to the drain line which flows through valves V6 and V16.

A second (redundant) pyrogen filter, PF2, through which dialysate that has first passed through PF1 must also pass before entering a patient's peritoneal cavity. PF2 is primed similarly to PF1 by opening valves V5 (to PF2) and V8 (to atmosphere) while valves V6 (to drain) and V9 (to patient fill) are closed to allow air to escape through a 0.2 micron sterilizing filter. The case of PF2 is primed by closing V8 and V9 and opening valves V11 (to patient drain and/or drain chamber) and V12 (to system drain) which allows fluid to fill the case and exit down the drain line through V16.

Pressure sensor P2 is located just upstream of PF1 and pressure sensor P3 is located downstream of PF1 between valves V4 and V5. Their purpose is to calculate and monitor the transmembrane pressure (TMP) across the fibers of PF1 to insure that the TMP never exceeds the maximum specified for this brand of pyrogen filter as indicated in the package insert for the product. Similarly, pressure sensor P4 is located just downstream of PF2 and upstream of valves V9 and V11 so that the TMP of PF2 can be calculated from P3 and P4.

Dialysate is directed to and from a patient through a tubing fluid path that is external to the dialysate solution preparation and treatment system. This fluid path is defined by the patient fill line and the patient drain line.

In addition to being used to calculate the TMP of PF2, P4 is also used to assure that the pressure of the dialysate flowing into the patient never exceeds the safe upper limit of positive pressure set in software.

It sometimes occurs that the indwelling peritoneal dialysis catheter terminating in the patient's peritoneal cavity becomes malpositioned or kinked, restricting the flow of dialysate. In order to detect this, a flow sensor FM1 is positioned upstream of the patient connect line (PCL). If the flow rate declines below an alarm limit set in software, or if P4 detects a sudden increase in pressure or both, an alarm will alert the patient to resolve the occlusion.

A patient drain line is connected to a mating receptacle on the face of the machine. This mating receptacle is, in turn, connected to a fluid path that flows through valve V10, flow sensor FM2, and into the drain chamber. Like the supply chamber, drain chamber is a flexible container that is surrounded by a rigid enclosure. The drain chamber, however, does not need to be vented to atmosphere since its expansion and collapse is controlled by directing either positive or negative air pressure from the positive and negative pressure reservoirs PPR and NPR through valves V13 and V14 respectively. The pressure residing in these reservoirs is controlled by pressure sensors P5 and P6 whose readings are monitored in software that dictates when to turn AC on or off in order to never exceed safe limits of positive and negative pressure as set in software. This assures that excessive pressure can never be exerted on the patient's peritoneum.

Flow sensor FM2 is used to monitor a restriction in the flow of dialysate from the patient during a drain phase caused by a kinked or occluded catheter, PCL or the patient drain line (PDL). If such a restriction is detected, dialysate flow will first be reversed from the drain chamber back into the patient's peritoneum in order to attempt to resolve the blockage without waking the patient. If after a series of reverse flows and forward flows fails to resolve the restriction, then an alarm will awaken the patient to change their body position.

Two flow paths are provided for the drainage of the drain chamber. One flow path is created by closing V10 and V11 and opening V12, and V16 which provides a direct path to drain. The other pathway is the one that is intended to be used during treatments. This flow path is created by closing V10 and V12 and opening V11, V6, and V16. By so doing, the dialysate that has been drained from the patient and into drain chamber is directed backwards through PF2 when positive pressure is applied to drain chamber. Since proteins, polypeptide chains of amino acids, and components of host defenses like white blood cells are too large to pass through the pyrogen filter membrane, they are caught on the patient-facing side of the membrane where they can then be reinfused into the patient with the following fill cycle.

A flow sensor, FM3, is positioned in the drain line between V6 and V16 in order to detect a restriction in flow during various phases of operation.

A conductivity sensor, C4, is positioned in the drain line upstream of V16 in order to detect air bubbles and to aid in assuring the quality of freshly made dialysate.

Valve V16 is a three-way valve that either directs flow directly to drain or through the disinfection bypass flow path in order to allow the recirculation of disinfectant through all flow paths.

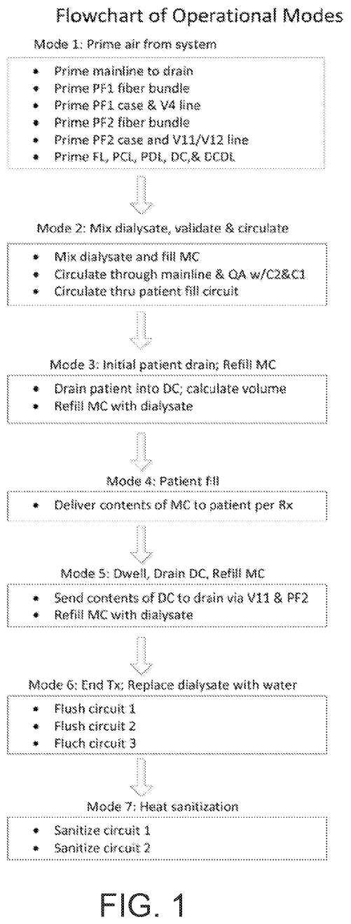

As shown in FIG. 1, a flow chart is provided which shows the sequence of the major modes of operation that the device will pass through as it performs a single peritoneal dialysis treatment.

The peritoneal dialysis solution preparation and treatment system is initially in a first phase of operation where it is assumed that this is the first operation of the system and it is filled with air necessitating the removal of the air. This is accomplished by perfusing highly purified water from a separate water purification system through a first flow path that includes V1, the mixing apparatus, the supply chamber, V3, V6, and the system drain line.

Next, the fiber bundle of the first pyrogen filter PF1 is primed by removing air through a 0.2 micron filter. That is, water is flowed through V1, the mixing apparatus, the supply chamber, V3, PF1, and V7.

Next, the extracapillary space within the case of PF1 is primed via a fluid path flowing through valves V4, V6, and V16.

Next, the fiber bundle of a second pyrogen filter, PF2, is primed by removing air through a 0.2 micron filter. That is, water is flowed through V1, the mixing apparatus, the supply chamber, V3, V5, PF2, and V8.

Next, the extracapillary space within the case of PF2 is primed via a fluid path flowing through valve V11 and V16.

Next, the tubing circuitry that allows for dialysate to enter and exit a patient is primed with water flowing from V1 through the mixing apparatus and the supply chamber, V3, V5, PF2, V9, FL, PCL, PDL, V10, the drain chamber, V12, and V16.

Next, peritoneal dialysate is formulated and mixed in the supply chamber by causing pumps PU1, PU2, PU3, and PU4 to meter into the supply chamber water, acid/electrolyte concentrate, neutralizing buffer, and osmotic concentrate respectively in proportion to the their concentration in the final dialysate prescription as recorded in integrated software.

Next, the composition and temperature of the dialysate being formulated in supply chamber is assured by allowing it to flow past temperature sensors T1 and T2 and conductivity sensors C2 and C3 via a flow path defined by V3, V6 and V16.

Next, the water previously used to prime the air out of the fluid paths is replaced by dialysate. This is accomplished by delivering the dialysate from the supply chamber through the flow path through both pyrogen filters and then through FL, PCL, PDL, the drain chamber, DCDL, V12 and finally through V16 and conductivity sensor C4. When the conductivity at C4 equals C2/C3, dialysate priming of the fluid paths is complete and the patient may be connected to PCL to initiate treatment.

Next, the patient connect line, PCL, is disconnected from its mating receptacle on the face of the instrument and connected to the patient's transfer set in preparation for the initiation of treatment whose first step is the draining of whatever residual dialysate is resident in the patient's peritoneum from the previous treatment. This is accomplished by opening V10 while V9, V11 and V12 are closed and applying negative air pressure to the rigid enclosure surrounding the flexible drain chamber, from the negative pressure reservoir, NPR, through valve V14. This will cause drain chamber to expand and draw dialysate out of the patient until the flow rate measured by FM2 approaches close to zero and the volume of dialysate removed as calculated by the integral of the FM2 flow rate over time falls within the expected range as recorded in a software lookup table. At the same time that the initial drain is occurring, the volume of dialysate in supply chamber can be topped up to replace that used to prime the fluid paths by causing pumps PU1, PU2, PU3, and PU4 to deliver their respective components into supply chamber as before until the volume of dialysate in supply chamber reaches the value set in the system software. The volume of dialysate delivered into supply chamber is calculated by summing the volumes delivered by each of the precise volumetric pumps PU1, PU2, PU3, and PU4.

Next, a volume of dialysate, as prescribed in the system software, is drained from the patient during treatment using the same flow path and calculation method as in the previous step the only difference being that generating more dialysate is not required in an intra-treatment drain phase as it will be typically accomplished during a dwell phase.

Next, during a drain phase, a restriction of flow is detected by the flow rate measured by FM2 falling below an alarm limit set in system software. This is an indication of the patient having rolled over on their tubing or the indwelling catheter having migrated into a position within the peritoneum that is restricting the outflow of dialysate. Before sounding an audible alarm to awaken the patient, the instrument will first attempt to resolve the occlusion by pushing previously drained dialysate back into the patient from the drain chamber. Should there be no dialysate residing in drain chamber when this condition occurs, then fresh dialysate from supply chamber will be instilled instead as illustrated in the following step. Such catheter unocclusion steps may be repeated multiple times over several minutes as set in system software preferences but if the restriction persists, then an audible alarm will be sounded.

Next, a prescribed volume of fresh dialysate in instilled into the patient's peritoneum. This is accomplished by applying positive air pressure to the rigid enclosure surrounding the supply chamber, by opening valve V15 to the positive pressure reservoir, PPR and closing valves V2, 6, 10, 11, and 12 while valves V3, 4, 5, and 9 are open. This causes dialysate to be delivered into the patient but only after first passing through pyrogen filters PF1 and PF2. The fill volume is assured by calculating the integral of the flow rate at FM1 over time.

Next, the dialysate instilled into the patient in the previous fill phase is allowed to dwell in the peritoneum for the prescribed number of minutes. During this dwell phase, spent dialysate residing in drain chamber from a previous drain phase is ejected down the drain line by closing V10 and opening V13 to PPR which causes the drain chamber to collapse. The flow path employed to eject the used dialysate is uniquely designed to cause this fluid, which contains beneficial biomolecules that are of a molecular size that is too large to pass through the membrane in PF2 and are desirably retained in the patients and components of host defense such as white blood cells whose conservation is highly desirable, to flow backwards through the capillary fibers of PF2 before being directed through valves V5, 6, 16 and down the drain. The aforementioned beneficial organic materials residing in the post-dwell period dialysate and which are too large to pass through the membrane are captured on the patient-facing side of the membrane and re-instilled into the patient with each next fill phase. Also, during a dwell phase, the next fill volume of fresh dialysate is mixed in supply chamber as before in preparation for the next fill phase.

Next, any dialysate remaining in the flow paths of the system after the end of a treatment when the patient has been disconnected (and PCL has been reattached to its mating receptacle on the face of the instrument) is flushed out completely by perfusing all flow paths with ultrapure water. This flushing is done in three phases. Water from the water purification system is drawn into the instrument through V1 by PU1 and flushed through V1, the mixing apparatus, the supply chamber, V3, V5, PF2, V11, V12, and V16. Not perfused in this phase are the flow paths between V3, PF1, V4, and P3, the conduit flowing through V11, and the conduit flowing through V6. The V11 segment is subsequently flushed and the V3-V6 segment is subsequently flushed by orienting the valves appropriately.

Next, the ultrapure water residing in all flow paths from the previous flushing phases is recirculated through by opening the disinfection bypass segment between three-way valves V1 and V16. During this disinfection phase, the heater, under control of temperature sensors T1, T2 and system software, heats the water such that the water entering the heater is maintained between 80 and 85 degrees centigrade for at least one hour. This assures that all flow paths will be subjected to high level disinfection allowing the patient connecting lines (FL, PCL, & PDL) to be used over multiple treatments and the internal flow paths to be devoid of any bacterial colonization. As in the previous flushing phases, not all flow paths can be disinfected simultaneously and valves must be oriented on an alternating basis so that the flow paths connected to the concentrate containers as well as the flow paths that pass though valves V3, V6 and V11 are also subjected to high level disinfection.

Next, the flow to and from the patient of injectable quality peritoneal dialysate made by the peritoneal dialysis solution preparation and treatment system is controlled by any commercially available peritoneal cycler. All such cyclers have associated tubing sets where one tube (the supply line) connects to the bag(s) of fresh dialysate and directs the fresh dialysate into the patient's peritoneum. Another tube (the drain line) directs the used dialysate from the patient to a drain bag or directly to a drain. In one of the current embodiments, the peritoneal dialysis solution preparation system replaces the supply bags and drain bags. Instead, the supply line and the drain line of a conventional cycler are connected directly into mating receptacles (SLC & DLC) on the face of the solution preparation device.

Because the cycler controls the flow of dialysate, there is no need for flow meter FM2, the drain chamber or the negative pressure reservoir NPR of the previous embodiment. In a first use scenario, air residing in the fluid paths and components of this embodiment would be primed just as in the 1st through the 6th phases of operation in the previous embodiment.

Next, a Disinfection Loop (DL) is attached between SLC and DLC at the end of the prior treatment thereby creating a closed loop between all of the fluid paths of the peritoneal dialysis solution preparation and treatment system. This configuration allows the dialysate remaining in the fluid paths from the prior treatment to be flushed out and replaced with ultrapure water and then subjected to hot water sanitization just as in previous phases of operation described above.

Next, a new batch of dialysate is mixed in supply chamber, its composition and temperature assured, and all fluid paths primed with dialysate in a manner similar to operational phases described above.

Next, the cycler is connected to the peritoneal dialysis solution preparation and treatment system and a volume of dialysate is filled into the patient under the control of the cycler. When the cycler attempts to draw fluid from the peritoneal dialysis solution preparation and treatment system, it will create a negative pressure which is sensed by pressure sensor P6. Once this negative pressure is identified by the system software, all valves will be oriented accordingly to allow dialysate residing in the supply chamber to be drawn through both depyrogenation filters, into the cycler, and then into patient.

A previous fill volume now resides in the patient and is allowed to dwell there according to the prescription resident in the cycler. During this phase, when there is no pressure being applied to either P6 or P7, the peritoneal dialysis solution preparation and treatment system prepares the next fill volume by metering the water and concentrates into the supply chamber and orienting all valves accordingly.

After the dwell time has elapsed, the cycler initiates a drain phase by pumping fluid out of the patient through the drain line DL. This causes a positive pressure to be registered at pressure sensor P7 which cause the software to orient the valves so as to permit the spent dialysate to pass through depyrogenation filter PF2 on its way to drain.

Once the final drain and fill are accomplished, the patient would disconnect the cycler tubing from the peritoneal dialysis solution preparation and treatment system and replace it with a disinfection loop DL. Then, either the patient will instruct the peritoneal dialysis solution preparation and treatment system to enter a rinse and disinfect mode by pressing a button on the use interface or the disinfection loop will be designed to be automatically sensed by the peritoneal dialysis solution preparation and treatment system which then trigger it to enter the rinse and disinfection mode in a manner similar to the previous embodiment.

### EMBODIMENTS OF THE DISCLOSURE

Embodiment 1: A Peritoneal Dialysis system produces injectable quality dialysate by proportioning near-zero conductivity water from a separate source with concentrates containing the requisite components commensurate with a physician's prescription, and perfusing the fully mixed and finally constituted dialysate through two depyrogenation filter in series before being instilled into a patient and, by, after every patient treatment, subjecting all fluid paths, including those that convey dialysate to and from a patient, to recirculating ultrapure water whose temperature is greater than 80 degrees centigrade for longer than one hour which results high level disinfection thereby reducing the possibility of bacterial colonization therein and allowing for the patient-connecting flow paths to be reused over multiple treatments.

Embodiment 2: The system of Embodiment 1, wherein the spent dialysate being drained from a patient is directed backwards through the second depyrogenation filter on its path to drain thereby capturing any organic matter that is too large to pass through the filter's membrane from where it can be reinfused into the patient on a subsequent fill cycle.

Embodiment 3: The system of Embodiment 1 wherein: the concentrate containing the required electrolytes and acid are provided in one container, a neutralizing buffer such as sodium bicarbonate is provided in a second container and concentrated glucose which has for example not been autoclaved but rather aseptically filled, is provided in a third container, and the contents of each container are metered into a stream of ultrapure water where the flow rate of each stream is precisely controlled such that the composition in the finally constituted dialysate can be customized by adjusting the proportion of each of the separate constituents by way of precisely controlling the flow rate of each metering pump, and if the glucose component is not autoclaved, its pH can be near neutral instead of in the range of 5.2 or lower and minimal, if not zero, glucose degradation products will be formed, and since the dialysate is being constituted immediately prior to infusion into a patient, sodium bicarbonate, for example, can be used as the buffering agent without any concerns of calcium and magnesium carbonate precipitates forming and a pH in the physiologic range (7.1-7.5) of the instilled dialysate can be achieved.

Embodiment 4: A peritoneal dialysis system wherein the chemical constituents of the peritoneal dialysate are metered into a flexible mixing chamber which is contained within a vented rigid outer housing that is sealed around the inner flexible portion. This chamber serves dual purposes; 1.) It provides a volume in which the chemical constituents of the peritoneal dialysate can be homogenously mixed together and, 2.) It serves as a pump to deliver its contents into a patient's peritoneal cavity. The flexible portion is allowed to expand under the positive pressure of the metering pumps delivering chemical constituents into it while the vent to atmosphere on the rigid outer portion is open and the exit conduit is occluded by a valve thereby allowing the water and chemicals to mix together. The contents of this flexible chamber are then delivered to the patient by closing the vent and the entrance to the flexible chamber while opening the valve at the exit of the chamber and subjecting the space between the rigid outer portion and the flexible chamber to positive air pressure generated by an air compressor. This positive air pressure causes the flexible portion to collapse and deliver the dialysate downstream.

As such, a system for peritoneal dialysis includes a mixing apparatus configured to mix water, an acid/electrolyte concentrate, an osmotic concentrate, and a neutralizing buffer to produce a supply dialysate. The system also includes a supply chamber configured to store the supply dialysate. The system also includes a first filter that includes a first port and a second port. The first filter is configured to receive the supply dialysate from the supply chamber at the first port and to remove contaminants (e.g., endotoxins and/or peptidoglycans) from the supply dialysate. The system also includes a second filter that includes a third port and a fourth port, a patient drain line connected to the third port, a patient fill line connected to the third port, and a system drain line connected to the fourth port. The system also includes one or more valves that: in a first configuration allow the second filter to receive a spent dialysate from the patient drain line at the third port to capture biological components (e.g., polypeptides, proteins, glycoproteins, white blood cells, macrophages, and/or chemotaxis components that tend to have molecular weights higher than that of uremic toxins) from the spent dialysate such that the spent dialysate is dispensed via the system drain line, and in a second configuration allow the second filter to receive the supply dialysate from the second port at the fourth port to remove further contaminants (e.g., endotoxins and/or peptidoglycans) from the supply dialysate such that the supply dialysate and the biological components are dispensed via the patient fill line (e.g., into the peritoneum of the patient).

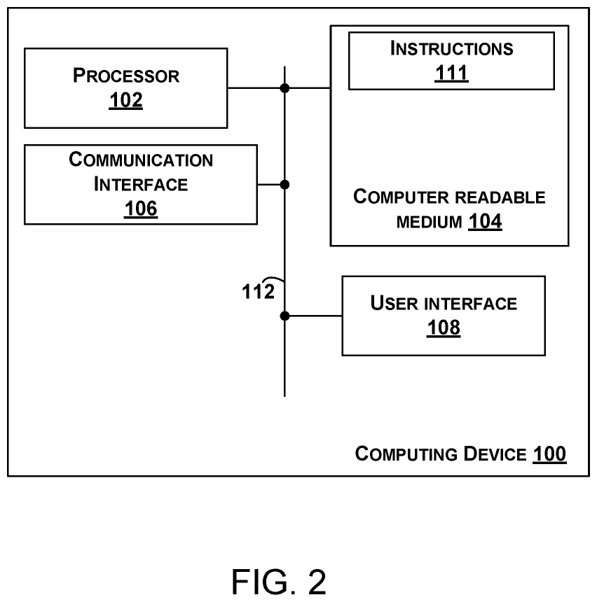

FIG. 2 is a block diagram of a computing device 100. The computing device 100 includes one or more processors 102, a non-transitory computer readable medium 104, a communication interface 106, and a user interface 108. Components of the computing device 100 are linked together by a system bus, network, or other connection mechanism 112.

The one or more processors 102 can be any type of processor(s), such as a microprocessor, a field programmable gate array, a digital signal processor, a multicore processor, etc., coupled to the non-transitory computer readable medium 104.

The non-transitory computer readable medium 104 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 104 can store instructions 111. The instructions 111 are executable by the one or more processors 102 to cause the computing device 100 to perform any of the functions or methods described herein.

The communication interface 106 can include hardware to enable communication within the computing device 100 and/or between the computing device 100 and one or more other devices. The hardware can include any type of input and/or output interfaces, a universal serial bus (USB), PCI Express, transmitters, receivers, and antennas, for example. The communication interface 106 can be configured to facilitate communication with one or more other devices, in accordance with one or more wired or wireless communication protocols. For example, the communication interface 106 can be configured to facilitate wireless data communication for the computing device 100 according to one or more wireless communication standards, such as one or more Institute of Electrical and Electronics Engineers (IEEE) 801.11 standards, ZigBee standards, Bluetooth standards, etc. As another example, the communication interface 106 can be configured to facilitate wired data communication with one or more other devices. The communication interface 106 can also include analog-to-digital converters (ADCs) or digital-to-analog converters (DACs) that the computing device 100 can use to control various components of the computing device 100 or external devices.

The user interface 108 can include any type of display component configured to display data. As one example, the user interface 108 can include a touchscreen display. As another example, the user interface 108 can include a flat-panel display, such as a liquid-crystal display (LCD) or a light-emitting diode (LED) display. The user interface 108 can include one or more pieces of hardware used to provide data and control signals to the computing device 100. For instance, the user interface 108 can include a mouse or a pointing device, a keyboard or a keypad, a microphone, a touchpad, or a touchscreen, among other possible types of user input devices. Generally, the user interface 108 can enable an operator to interact with a graphical user interface (GUI) provided by the computing device 100 (e.g., displayed by the user interface 108).

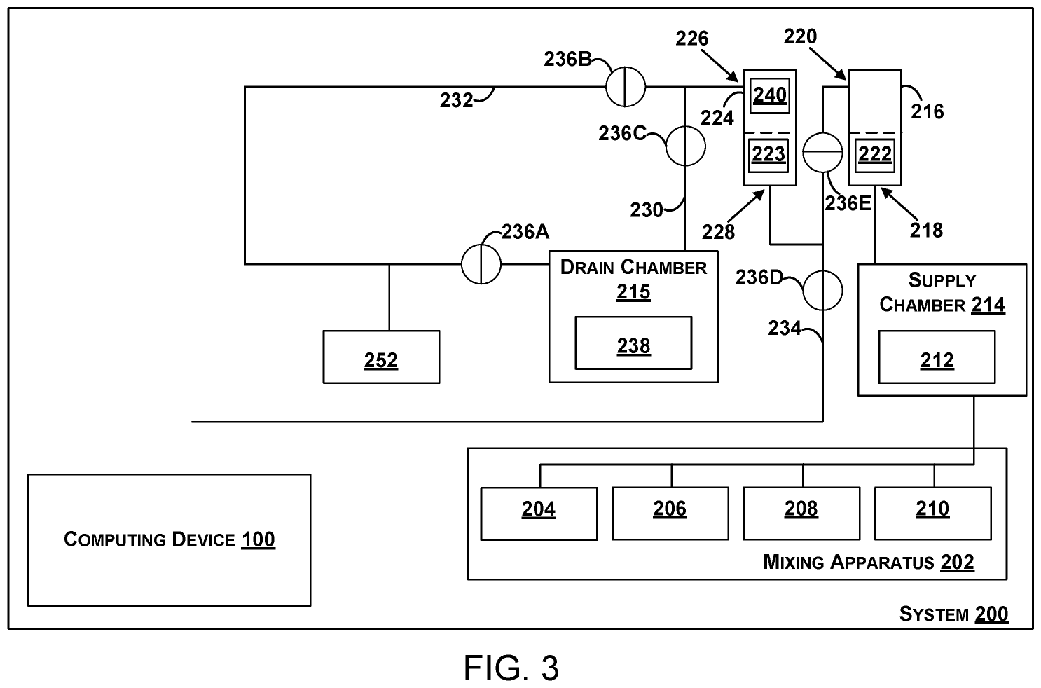

FIG. 3 is a schematic diagram of a system 200 for peritoneal dialysis. The system 200 includes a mixing apparatus 202 configured to mix water 204, an acid/electrolyte concentrate 206, an osmotic concentrate 208, and a neutralizing buffer 210 to produce a supply dialysate 212. The system 200 also includes a supply chamber 214 configured to store the supply dialysate 212. The system 200 also includes a filter 216 that includes a port 218 and a port 220. The filter 216 is configured to receive the supply dialysate 212 from the supply chamber 214 at the port 218 and to remove contaminants 222 from the supply dialysate 212. The system 200 also includes a filter 224 that includes a port 226 and a port 228, a patient drain line 230 connected to the port 226, a patient fill line 232 connected to the port 226, and a system drain line 234 connected to the port 228. The system 200 also includes a valve 236A, a valve 236B, a valve 236C, a valve 236D, and a valve 236E that, in the configuration shown in FIG. 3, allow the filter 224 to receive a spent dialysate 238 from the patient drain line 230 at the port 226 to capture biological components 240 (e.g., proteins and host defenses such as white blood cells, macrophages, and/or chemotaxis components) from the spent dialysate 238 such that the spent dialysate 238 is dispensed via the system drain line 234. The various components of the system 200 can be in fluid communication with each other via flexible tubing or other known means for transporting fluid.

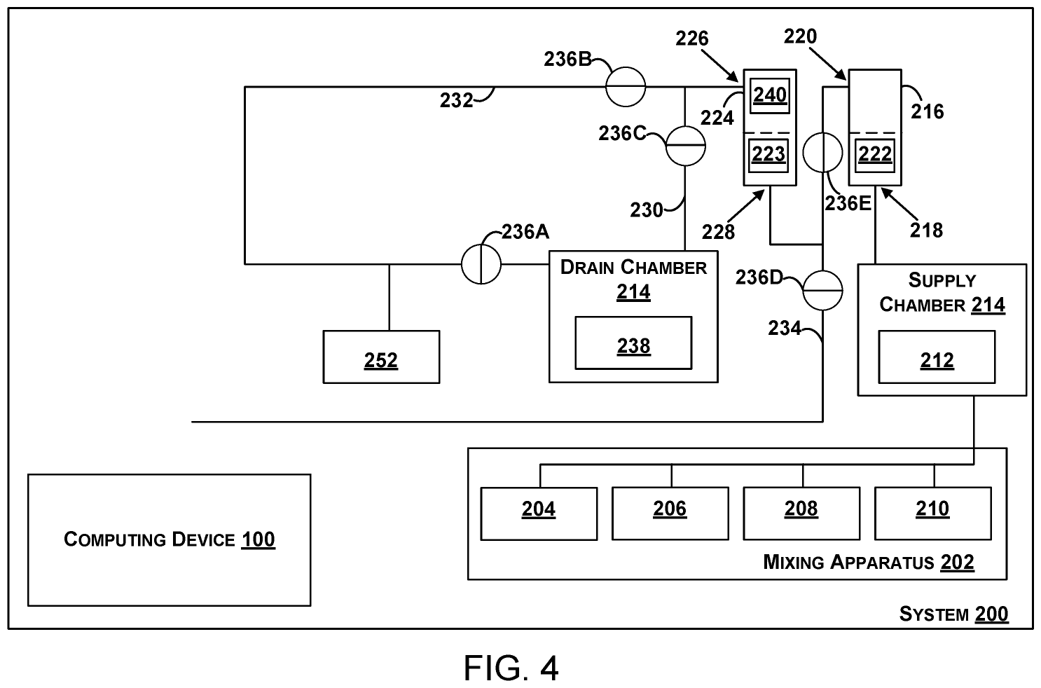

In a configuration shown in FIG. 4 and discussed further below, the valves 236A-E are in a configuration to allow the filter 224 to receive the supply dialysate 212 from the port 220 at the port 228 to remove further contaminants 223 from the supply dialysate 212 such that the supply dialysate 212 and the biological components 240 are dispensed via the patient fill line 232.

Referring back to FIG. 3, the mixing apparatus 202 is typically configured to receive the water 204 from a source external to the system 200, such as a reverse osmosis purification system. The mixing apparatus 202 includes a positive displacement metering pump configured to provide a controlled amount of the water 204 to the supply chamber 214.

The mixing apparatus 202 also includes a proportioning pump configured to provide a controlled amount of the acid/electrolyte concentrate 206 to the supply chamber 214. The acid/electrolyte concentrate 206 includes one or more of water, anhydrous glucose, sodium, calcium, magnesium, chloride, bicarbonate, and/or lactate. The electrolyte component of the acid/electrolyte concentrate 206 generally ends up being about 140 (mmol/L) of sodium, 2.5 mmol/L of calcium, or 0.75 mmol/L of magnesium, for example.

The mixing apparatus 202 also includes a proportioning pump configured to provide a controlled amount of the osmotic concentrate 208 to the supply chamber 214. The osmotic concentrate 208 includes water, glucose (e.g., aseptically filled glucose and/or dextrorotatory glucose), and/or icodextrin.

The mixing apparatus 202 also includes a proportioning pump configured to provide a controlled amount of the neutralizing buffer 210 to the supply chamber 214. The neutralizing buffer 210 includes water, and a neutralizing base such as sodium bicarbonate, sodium lactate, and/or sodium carbonate.

The supply chamber 214 includes a rigid housing and a flexible container (e.g., a plastic bag) disposed within the rigid housing. The flexible container is configured to store the supply dialysate 212 and the rigid housing is configured to apply pressure to compress or expand the flexible container, thereby expelling the supply dialysate 212 from the flexible container into the filter 216 or drawing the supply dialysate 212 into the flexible container from the mixing apparatus 202, respectively.

The filter 216 and the filter 224 generally take the form of depyrogenation filters configured to retain the contaminants 222 and the further contaminants 223 in the form of endotoxins and peptidoglycans. In some examples, the filter 216 and the filter 224 have a molecular weight cut off (MWCO) defined by a 90% retention rate within a range of 50 Daltons to 80,000 Daltons.

The patient drain line 230, the patient fill line 232, and the system drain line 234 can take the form of flexible tubing, but other examples are possible.

FIG. 3 shows the system 200 in a state after removal of at least some of the spent dialysate 238 from the patient's peritoneal cavity 252. The spent dialysate 238 was previously the supply dialysate 212, but after perhaps several hours of dwell time in the peritoneal cavity 252, the supply dialysate 212 absorbed toxins and water that crossed the patient's peritoneal membrane into the peritoneal cavity 252 and became the spent dialysate 238.

In a step previous to what is shown in FIG. 3, the valve 236A can be opened and the valve 236B and the valve 236C can be closed so that the drain chamber 215 can receive the spent dialysate 238 from the peritoneal cavity 252 (e.g., via a catheter). The spent dialysate 238 can be moved into the drain chamber 215 via gravity and/or the drain chamber 215 can be operated to apply negative pressure that draws the spent dialysate 238 into the drain chamber 215.

In some examples, the drain chamber 215 is not included as part of the system 200 and instead a peritoneal dialysis cycler is used. For example, the system 200 receives the spent dialysate 238 directly from the peritoneal dialysis cycler and the spent dialysate 238 is moved through the valve 236C and the filter 224 as described above.

As shown in FIG. 3, the valve 236A is then closed and the valve 236C is opened and the system 200 moves the spent dialysate 238 from the drain chamber 215 (or from the peritoneal dialysis cycler) into the port 226 via the valve 236C and then through the filter 224 to capture the biological components 240 from the spent dialysate 238. The system 200 then dispenses (e.g., disposes) the spent dialysate 238 (e.g., along with uremic toxins that passed through the filter 224) via the system drain line 234 through the valve 236D. The spent dialysate 238 can be moved via positive pressure applied to the drain chamber 215 or by operation of the peritoneal dialysis cycler. This process generates depleted spent dialysate 238 with the biological components 240 removed, which is disposed via the system drain line 234.

FIG. 4 is a schematic diagram of the system 200 in a configuration for removing the contaminants 222 and the further contaminants 223 from the supply dialysate 212 such that the supply dialysate 212 and the captured biological components 240 are dispensed via the patient fill line 232 into the peritoneal cavity 252.

Prior to, concurrent with, or after the filter 224 captures the biological components 240, the system 200 mixes, via the mixing apparatus 202, the water 204, the acid/electrolyte concentrate 206, the osmotic concentrate 208, and the neutralizing buffer 210 to produce the supply dialysate 212. For example, controlled amounts of the water 204, the acid/electrolyte concentrate 206, the osmotic concentrate 208, and the neutralizing buffer 210 are introduced into the supply chamber 214 such that the supply dialysate 212 becomes substantially homogeneous within the supply chamber 214. The mixing can take place at room temperature, for example, at a temperature less than 100° F. and at a potential of hydrogen (pH) that is greater than 7.1 and less than 7.5.

After the biological components 240 have been captured by the filter 224 (as described above with reference to FIG. 3), the system 200 moves the supply dialysate 212, via positive pressure applied to the supply chamber 214, into the port 218 so that the filter 216 removes the contaminants 222 from the supply dialysate 212. The valve 236E is opened and the valve 236D is closed so that the supply dialysate 212 moves from the port 220 into the port 228 of the filter 224. The filter 224 removes the further contaminants 223 from the supply dialysate 212. The system 200 then dispenses the supply dialysate 212 and the biological components 240 from the port 226 via the patient fill line 232 into the peritoneal cavity 252 using the positive pressure applied to the supply chamber 214. The purified supply dialysate 212 can dwell in the peritoneal cavity 252 for several hours to facilitate removal of toxins from the patient's blood via the peritoneal membrane. This process results in the generation of the spent dialysate 238 that contains the toxins. This cycle of removing the spent dialysate 238 from the peritoneal cavity 252, using the filter 224 to capture the biological components 240 within the spent dialysate 238, and reintroducing the supply dialysate 212 and the biological components 240 back into the peritoneal cavity 252 can be repeated several times.

In some examples, the system 200 has various interlock features. For example, the system 200 can make a determination that the water 204 received by the mixing apparatus 202 has an electrical conductivity that is less than a threshold value (e.g., 0.055 μS/cm+/−10%) and mix the water 204, the acid/electrolyte concentrate 206, the osmotic concentrate 208, and the neutralizing buffer 210 in response to making the determination. The conductivity can be detected using a sensor that is configured to sample the water 204 as it enters the mixing apparatus 202. The water 204 should have a low conductivity that corresponds with high purity, for the benefit of the patient.

In a similar fashion, the system 200 can make a determination that the supply dialysate 212 has an electrical conductivity that is greater than a first threshold value and less than a second threshold value (e.g., within a desired range such as 12.2±0.2 ms/cm). The system 200 can move the supply dialysate 212 into the port 218 in response to making the determination. The supply dialysate 212 should have a conductivity that corresponds with a desired range, for the benefit of the patient.

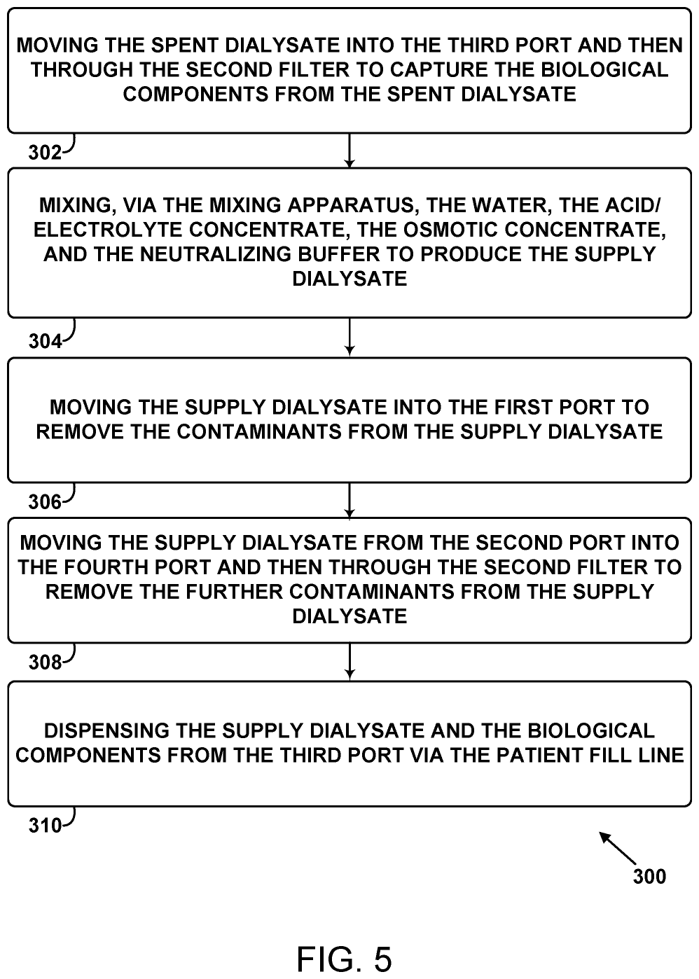

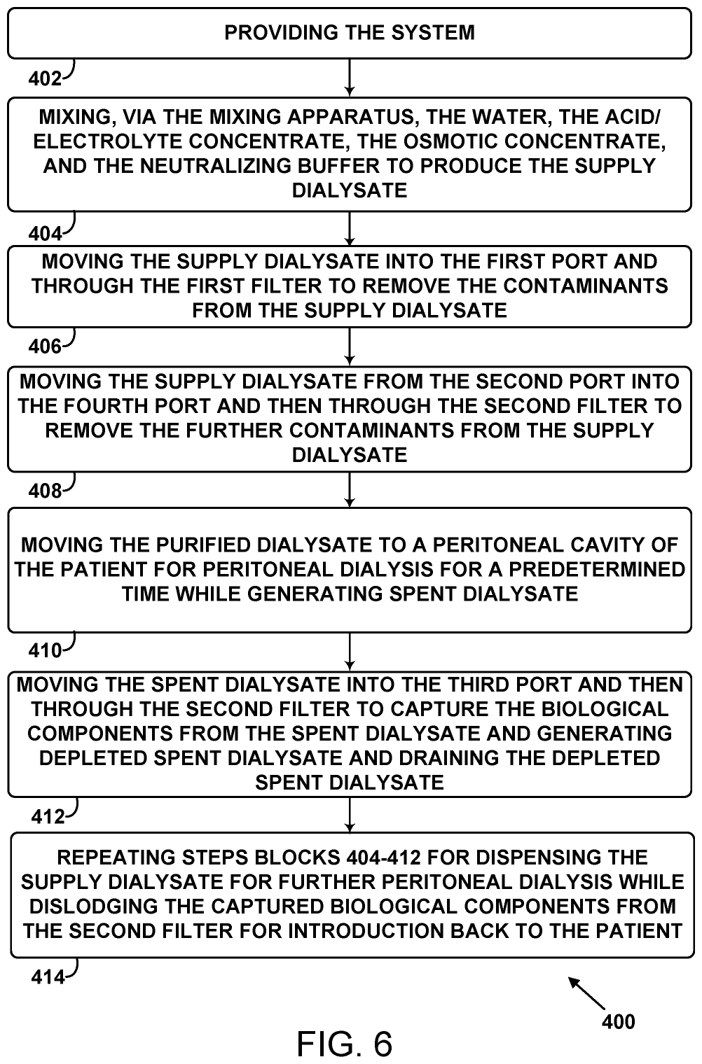

FIG. 5 and FIG. 6 are block diagrams of a method 300 and a method 400, which in some examples are performed by the system 200 and/or manually. As shown in FIG. 5 and FIG. 6, the method 300 and the method 400 include one or more operations, functions, or actions as illustrated by blocks 302, 304, 306, 308, 310, 402, 404, 406, 408, 410, 412, and 414. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 302, the method 300 includes moving the spent dialysate 238 into the port 226 and then through the filter 224 to capture the biological components 240 from the spent dialysate 238. Functionality related to block 302 is described above with reference to FIG. 3.

At block 304, the method 300 includes mixing, via the mixing apparatus 202, the water 204, the acid/electrolyte concentrate 206, the osmotic concentrate 208, and the neutralizing buffer 210 to produce the supply dialysate 212. Functionality related to block 304 is described above with reference to FIG. 4.

At block 306, the method 300 includes moving the supply dialysate 212 into the port 218 to remove the contaminants 222 from the supply dialysate 212. Functionality related to block 306 is described above with reference to FIG. 4.

At block 308, the method 300 includes moving the supply dialysate 212 from the port 220 into the port 228 and then through the filter 224 to remove the further contaminants 223 from the supply dialysate 212. Functionality related to block 308 is described above with reference to FIG. 4.

At block 310, the method 300 includes dispensing the supply dialysate 212 and the biological components 240 from the port 226 via the patient fill line 232. Functionality related to block 310 is described above with reference to FIG. 4.

At block 402, the method 400 includes providing the system 200. Functionality related to block 402 is described above with reference to FIG. 3 and FIG. 4.

At block 404, the method 400 includes mixing, via the mixing apparatus 202, the water 204, the acid/electrolyte concentrate 206, the osmotic concentrate 208, and the neutralizing buffer 210 to produce the supply dialysate 212. Functionality related to block 404 is described above with reference to FIG. 4.

At block 406, the method 400 includes moving the supply dialysate 212 into the port 218 and through the filter 216 to remove the contaminants 222 from the supply dialysate 212. Functionality related to block 406 is described above with reference to FIG. 4.

At block 408, the method 400 includes moving the supply dialysate 212 from the port 220 into the port 228 and then through the filter 224 to remove the further contaminants 223 from the supply dialysate 212. Functionality related to block 408 is described above with reference to FIG. 4.

At block 410, the method 400 includes moving the purified dialysate 212 to a peritoneal cavity 252 of the patient for peritoneal dialysis for a predetermined time while generating spent dialysate 238. Functionality related to block 410 is described above with reference to FIG. 4.

At block 412, the method 400 includes moving the spent dialysate 238 into the port 226 and then through the filter 224 to capture the biological components 240 from the spent dialysate 238 and generating depleted spent dialysate 238 and draining the depleted spent dialysate 238. Functionality related to block 410 is described above with reference to FIG. 3.

At block 414, the method 400 includes repeating blocks 404-412 for dispensing the supply dialysate 212 for further peritoneal dialysis while dislodging the captured biological components 240 from the filter 224 for introduction back to the patient. Functionality related to block 410 is described above with reference to FIG. 3 and FIG. 4.

### FURTHER EXAMPLE EMBODIMENTS

Example 1 is a system for peritoneal dialysis, the system comprising: a mixing apparatus configured to mix water, an acid/electrolyte concentrate, an osmotic concentrate, and a neutralizing buffer to produce a supply dialysate; a supply chamber configured to store the supply dialysate; a first filter comprising a first port and a second port, wherein the first filter is configured to receive the supply dialysate from the supply chamber at the first port and to remove contaminants from the supply dialysate; a second filter comprising a third port and a fourth port; a patient drain line connected to the third port; a patient fill line connected to the third port; a system drain line connected to the fourth port; and one or more valves that: in a first configuration allow the second filter to receive a spent dialysate from the patient drain line at the third port to capture biological components from the spent dialysate such that the spent dialysate is dispensed via the system drain line, and in a second configuration allow the second filter to receive the supply dialysate from the second port at the fourth port to remove further contaminants from the supply dialysate such that the supply dialysate and the biological components are dispensed via the patient fill line.

Example 2 is the system of example 1, wherein the mixing apparatus is configured to receive the water from a source external to the system.

Example 3 is the system of any one of examples 1-2, wherein the mixing apparatus comprises a positive displacement metering pump configured to provide a controlled amount of the water to the supply chamber.

Example 4 is the system of any one of examples 1-3, wherein the mixing apparatus comprises a proportioning pump configured to provide a controlled amount of the acid/electrolyte concentrate to the supply chamber.

Example 5 is the system of any one of examples 1-4, wherein the mixing apparatus comprises a proportioning pump configured to provide a controlled amount of the osmotic concentrate to the supply chamber.

Example 6 is the system of any one of examples 1-5, wherein the mixing apparatus comprises a proportioning pump configured to provide a controlled amount of the neutralizing buffer to the supply chamber.

Example 7 is the system of any one of examples 1-6, wherein the supply chamber comprises: a rigid housing; and a flexible container disposed within the rigid housing, wherein the flexible container is configured to store the supply dialysate and the rigid housing is configured to apply pressure to compress or expand the flexible container, thereby expelling the supply dialysate from the flexible container or drawing the supply dialysate into the flexible container.

Example 8 is the system of any one of examples 1-7, wherein the first filter and/or the second filter comprises a depyrogenation filter.

Example 9 is the system of any one of examples 1-8, wherein the first filter and/or the second filter has a molecular weight cut off (MWCO) defined by a 90% retention rate within a range of 50 Daltons to 80,000 Daltons and a pyrogen removal factor of 105 or greater.

Example 10 is the system of any one of examples 1-9, wherein the first filter and the second filter are configured to retain endotoxins and peptidoglycans.

Example 11 is a method of operating the system of any one of examples 1-10, the method comprising: moving the spent dialysate into the third port and then through the second filter to capture the biological components from the spent dialysate; mixing, via the mixing apparatus, the water, the acid/electrolyte concentrate, the osmotic concentrate, and the neutralizing buffer to produce the supply dialysate; moving the supply dialysate into the first port to remove the contaminants from the supply dialysate; moving the supply dialysate from the second port into the fourth port and then through the second filter to remove the further contaminants from the supply dialysate; and dispensing the supply dialysate and the biological components from the third port via the patient fill line.

Example 12 is the method of example 11, further comprising dispensing the spent dialysate via the system drain line.

Example 13 is the method of any one of examples 11-12, further comprising receiving the spent dialysate from a peritoneum of a patient prior to moving the spent dialysate into the third port.

Example 14 is the method of example 13, wherein dispensing the supply dialysate and the biological components comprises dispensing the supply dialysate and the biological components into the peritoneum.

Example 15 is the method of any one of examples 11-14, further comprising receiving the spent dialysate from a peritoneal dialysis cycler prior to moving the spent dialysate into the third port.

Example 16 is the method of example 15, wherein dispensing the supply dialysate and the biological components comprises dispensing the supply dialysate and the biological components into the peritoneal dialysis cycler.

Example 17 is the method of any one of examples 11-16, wherein the neutralizing buffer comprises sodium bicarbonate, sodium lactate, and/or sodium carbonate.

Example 18 is the method of any one of examples 11-17, wherein the osmotic concentrate comprises glucose and/or icodextrin.

Example 19 is the method of any one of examples 11-18, wherein the contaminants comprise endotoxins and/or peptidoglycans.

Example 20 is the method of any one of examples 11-19, wherein the acid/electrolyte concentrate comprises anhydrous glucose, sodium, calcium, magnesium, chloride, bicarbonate, and/or lactate.

Example 21 is the method of any one of examples 11-20, further comprising: making a determination that the water has an electrical conductivity that is less than a threshold value, wherein the mixing is performed in response to making the determination.

Example 22 is the method of any one of examples 11-21, further comprising: making a determination that the supply dialysate has an electrical conductivity that is greater than a first threshold value and less than a second threshold value, wherein the moving the supply dialysate into the first port is performed in response to making the determination.

Example 23 is the method of any one of examples 11-22, wherein the mixing comprises mixing such that the supply dialysate becomes substantially homogeneous within the supply chamber.

Example 24 is the method of any one of examples 11-23, wherein the mixing comprises mixing while the water, the acid/electrolyte concentrate, the osmotic concentrate, and the neutralizing buffer are at a temperature of less than 100° F.

Example 25 is the method of any one of examples 11-24, wherein the osmotic concentrate comprises dextrorotatory glucose.

Example 26 is the method of any one of examples 11-25, wherein the osmotic concentrate comprises aseptically filled glucose.

Example 27 is the method of any one of examples 11-26, wherein the mixing comprises mixing at a potential of hydrogen (pH) that is greater than 7.1 and less than 7.5.

Example 28 is the system of any one of examples 1-10, further comprising: one or more processors; and a computer readable medium storing instructions that, when executed by the one or more processors, cause the system to perform the method of any one of examples 11-25.

Example 29 is a computer readable medium storing instructions that, when executed by the system of example 28, cause the system to perform the method of any one of examples 11-27.

Example 30 is a system for peritoneal dialysis, the system comprising: a mixing apparatus configured to mix water, an acid/electrolyte concentrate, a osmotic concentrate, and a neutralizing buffer to produce a supply dialysate; a supply chamber configured to store the supply dialysate; a drain line; a filter; and one or more valves that in a first configuration allow the filter to receive a spent dialysate from the drain line at a first port of the filter to capture biological components within the spent dialysate and in a second configuration allow the filter to receive the supply dialysate from the supply chamber at a second port of the filter to remove contaminants from the supply dialysate.

Example 31 is a method for conducting peritoneal dialysis, the method comprising: providing the system of any one of examples 1-10; mixing, via the mixing apparatus, the water, the acid/electrolyte concentrate, the osmotic concentrate, and the neutralizing buffer to produce the supply dialysate; moving the supply dialysate into the first port and through the first filter to remove the contaminants from the supply dialysate; moving the supply dialysate from the second port into the fourth port and then through the second filter to remove the further contaminants from the supply dialysate; moving the purified dialysate to a peritoneal cavity of the patient for peritoneal dialysis for a predetermined time while generating spent dialysate; moving the spent dialysate into the third port and then through the second filter to capture the biological components from the spent dialysate and generating depleted spent dialysate and draining the depleted spent dialysate; and repeating steps (ii) to (vi) for dispensing the supply dialysate for further peritoneal dialysis while dislodging the captured biological components from the second filter for introduction back to the patient.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1. A system for peritoneal dialysis, the system comprising:
a mixing apparatus configured to mix water, an acid/electrolyte concentrate, an osmotic concentrate, and a neutralizing buffer to produce a supply dialysate;
a supply chamber configured to store the supply dialysate;
a first filter comprising a first port and a second port, wherein the first filter is configured to receive the supply dialysate from the supply chamber at the first port and to remove contaminants from the supply dialysate;
a second filter comprising a third port and a fourth port;
a patient drain line connected to the third port;
a patient fill line connected to the third port;
a system drain line connected to the fourth port; and
one or more valves that:
in a first configuration allow the second filter to receive a spent dialysate from the patient drain line at the third port to capture biological components from the spent dialysate such that the spent dialysate is dispensed via the system drain line, and
in a second configuration allow the second filter to receive the supply dialysate from the second port at the fourth port to remove further contaminants from the supply dialysate such that the supply dialysate and the biological components are dispensed via the patient fill line.

2-6. (canceled)

7. The system of claim 1, wherein the supply chamber comprises:
a rigid housing; and
a flexible container disposed within the rigid housing, wherein the flexible container is configured to store the supply dialysate and the rigid housing is configured to apply pressure to compress or expand the flexible container, thereby expelling the supply dialysate from the flexible container or drawing the supply dialysate into the flexible container.

8. (canceled)

9. The system of claim 1, wherein the first filter and/or the second filter has a molecular weight cut off (MWCO) defined by a 90% retention rate within a range of 50 Daltons to 80,000 Daltons and a pyrogen removal factor of 105 or greater.

10. The system of claim 1, wherein the first filter and the second filter are configured to retain endotoxins and peptidoglycans.

11. A method comprising:
moving spent dialysate into a third port of a second filter and then through the second filter to capture the biological components from the spent dialysate;
mixing, via a mixing apparatus, water, an acid/electrolyte concentrate, an osmotic concentrate, and a neutralizing buffer to produce supply dialysate;
moving the supply dialysate into a first port of a first filter to remove contaminants from the supply dialysate;
moving the supply dialysate from the second port of the first filter into a fourth port of the second filter and then through the second filter to remove the further contaminants from the supply dialysate; and
dispensing the supply dialysate and the biological components from the third port via a patient fill line.

12. (canceled)

13. The method of claim 11, further comprising receiving the spent dialysate from a peritoneum of a patient prior to moving the spent dialysate into the third port.

14. The method of claim 13, wherein dispensing the supply dialysate and the biological components comprises dispensing the supply dialysate and the biological components into the peritoneum.

15. The method of claim 11, further comprising receiving the spent dialysate from a peritoneal dialysis cycler prior to moving the spent dialysate into the third port.

16. The method of claim 15, wherein dispensing the supply dialysate and the biological components comprises dispensing the supply dialysate and the biological components into the peritoneal dialysis cycler.

17-18. (canceled)

19. The method of claim 11, wherein
the contaminants comprise endotoxins and/or peptidoglycans.

20. (canceled)

21. The method of claim 11, further comprising:
making a determination that the water has an electrical conductivity that is less than a threshold value,
wherein the mixing is performed in response to making the determination.

22. The method of claim 11, further comprising:
making a determination that the supply dialysate has an electrical conductivity that is greater than a first threshold value and less than a second threshold value,
wherein the moving the supply dialysate into the first port is performed in response to making the determination.

23. The method of claim 11, wherein the mixing comprises mixing such that the supply dialysate becomes substantially homogeneous within a supply chamber.

24. The method of claim 11, wherein the mixing comprises mixing while the water, the acid/electrolyte concentrate, the osmotic concentrate, and the neutralizing buffer are at a temperature of less than 100° F.

25. The method of claim 11, wherein the osmotic concentrate comprises dextrorotatory glucose.

26. The method of claim 11, wherein the osmotic concentrate comprises aseptically filled glucose.

27. The method of claim 11, wherein the mixing comprises mixing at a potential of hydrogen (pH) that is greater than 7.1 and less than 7.5.

28. (canceled)

29. A computer readable medium storing instructions that, when executed by a system, cause the system to perform the method of claim 1.

30. A system for peritoneal dialysis, the system comprising:
a mixing apparatus configured to mix water, an acid/electrolyte concentrate, a osmotic concentrate, and a neutralizing buffer to produce a supply dialysate;
a supply chamber configured to store the supply dialysate;
a drain line;
a filter; and
one or more valves that in a first configuration allow the filter to receive a spent dialysate from the drain line at a first port of the filter to capture biological components within the spent dialysate and in a second configuration allow the filter to receive the supply dialysate from the supply chamber at a second port of the filter to remove contaminants from the supply dialysate.

31. A method for conducting peritoneal dialysis, the method comprising:
(i) providing the system of claim 1;
(ii) mixing, via the mixing apparatus, the water, the acid/electrolyte concentrate, the osmotic concentrate, and the neutralizing buffer to produce the supply dialysate;
(iii) moving the supply dialysate into the first port and through the first filter to remove the contaminants from the supply dialysate;
(iv) moving the supply dialysate from the second port into the fourth port and then through the second filter to remove further contaminants from the supply dialysate, thereby producing purified dialysate;
(v) moving the purified dialysate to a peritoneal cavity of the patient for peritoneal dialysis for a predetermined time while generating spent dialysate;
(vi) moving the spent dialysate into the third port and then through the second filter to capture the biological components from the spent dialysate and generating depleted spent dialysate and draining the depleted spent dialysate; and
(vii) repeating steps (ii) to (vi) for dispensing the supply dialysate for further peritoneal dialysis while dislodging the biological components from the second filter for introduction back to the patient.
